(19) United States
(12) Patent Application Publication
(10) Pub. No.: US 2024/0299276 A1
(43) Pub. Date: Sep. 12, 2024

(54) COMPOSITIONS AND METHODS FOR TREATING AND PREVENTING SKIN DISORDERS AND METHODS OF MANUFACTURE AND USE THEREOF

(71) Applicant: AlphaOlympia LLC, Villanova, PA (US)

(72) Inventors: Cynthia KUPER, Villanova, PA (US); Konstantinos LAHANAS, Ramsey, NJ (US)

(21) Appl. No.: 18/562,701

(22) PCT Filed: May 19, 2022
(86) PCT No.: PCT/US2022/030084

§ 371 Date: Nov. 20, 2023

Related U.S. Application Data
(60) Provisional application No. 63/190,550, filed on May 19, 2021.

(51) Int. Cl. A61K 8/60; A61K 8/34; A61K 8/67; A61K 8/9789; A61Q 19/00

(52) U.S. Cl. CPC A61K 8/60; A61K 8/347; A61K 8/67; A61K 8/676; A61K 8/9789; A61Q 19/00

(57) ABSTRACT

The invention encompasses compositions and methods for treating skin and preventing disorders of the skin. In various embodiments, compositions of the invention enhance cell turn over, moisture balance, decrease modalities of inflammation, bacterial adhesion, and oxidation of sebum in a hydrogel polymer carrier system for comedogenic and acnegenic prevention especially to pre-adolescents.

### CROSS-REFERENCE TO RELATED APPLICATIONS

This application is a U.S. National Stage Application under 35 U.S.C. § 371 of International Application No. PCT/US2022/030084, which claims priority to U.S. Provisional Application No. 63/190,550, filed May 19, 2021, the disclosures of which are hereby incorporated by reference in their entireties for all purposes.

### FIELD OF THE INVENTION

The invention encompasses compositions and methods for treating skin and preventing disorders of the skin. In various embodiments, compositions of the invention enhance cell turn over, moisture balance, decrease modalities of inflammation, bacterial adhesion, and oxidation of sebum in a hydrogel polymer carrier system for comedogenic and acnegenic prevention especially to pre-adolescents.

### BACKGROUND OF THE INVENTION

The claimed compositions and methods encompass treatments and modes of prevention of skin problems for the younger pre-adolescent generations through novel skin care products, whether they be novel in their combination of known ingredients, their modes of application or their engaging appearance. The compositions and methods of the invention target a young person's skin for early treatment and prevention and adoption of good hygiene and personal care habits as early as possible.

### SUMMARY OF THE INVENTION

The invention also encompasses formulations for the treatment and prevention intended for preadolescents focusing on skin care for many of its vulnerabilities, and a way to strengthen the skin health throughout adolescent changes in hormones, oil production, free radical exposure and expression of genetically mediated skin disease and inflammatory processes.

In general the invention encompasses a skin care composition comprising:

N-acetylglucosamine (NAG), cholecalciferol, tetrahexyldecyl ascorbate, resveratrol, white tea leaf extract, and an anionic polysaccharide.

In certain embodiments, the N-acetyl-glucosamine is present in an amount of about 1% (w/w) to about 5% (w/w) based on the total weight of the composition.

In certain embodiments, the cholecalciferol is present in an amount of about 0.1% (w/w) to about 5% (w/w) based on the total weight of the composition.

In certain embodiments, the tetrahexyldecyl ascorbate is present in an amount of about 0.1% (w/w) to about 5% (w/w) based on the total weight of the composition.

In certain embodiments, the resveratrol is present in an amount of about 0.1% (w/w) to about 10% (w/w) based on the total weight of the composition.

In certain embodiments, the white tea leaf extract is present in an amount of about 0.1% (w/w) to about 10% (w/w) based on the total weight of the composition.

In certain embodiments, the linear anionic polysaccharide is

present in an amount of about 0.1% (w/w) to about 5% (w/w) based on the total weight of the composition.

In certain embodiments, the amount of NAG is about 1.0% (w/w) based on the total weight of the composition.

In certain embodiments, the amount of cholecalciferol is about 0.1% (w/w) based on the total weight of the composition.

In certain embodiments, the amount of tetrahexyldecyl ascorbate is about 0.1% (w/w) based on the total weight of the composition.

In certain embodiments, the amount of resveratrol is about 2.0% (w/w) based on the total weight of the composition.

In certain embodiments, the amount of white tea leaf extract is about 2.0% (w/w) based on the total weight of the composition.

In certain embodiments, the amount of linear anionic polysaccharide is about 2.0% (w/w) based on the total weight of the composition.

In certain embodiments, the linear anionic polysaccharide is biosaccharide gum-1.

In certain embodiments, the skin care composition includes the following components:
(i) about 1.0% (w/w) NAG;
(ii) about 0.1% (w/w) cholecalciferol;
(iii) about 0.1% (w/w) tetrahexyldecyl ascorbate;
(iv) about 2.0% (w/w) resveratrol;
(v) about 2.0% (w/w) tea leaf extract; and
(vi) about 2.0% (w/w) anionic polysaccharide.

In other embodiments, the invention encompasses a method of treating or preventing a skin condition comprising administering a skin care composition or formulation as disclosed herein.

### DETAILED DESCRIPTION OF THE INVENTION

The invention generally encompasses skin care compositions and formulations for treating disorders of the skin. The compositions include n-acetylglucosamine, cholecalciferol, tetrahexyldecyl ascorbate, resveratrol white tea leaf extract, and a linear anionic polysaccharide (e.g., fucogel) in amounts effective to treat or prevent skin disorders.

In certain embodiments, the invention encompasses a composition including a combination of agents including, but not limited to, and in varying percentages by weight of n-acetylglucosamine, (NAG), along with other saccharide molecules of similar biologic composition, along with cholecalciferol in various suspended forms, tetrahexyldecyl ascorbate, resveratrol derived from a variety of sources, white tea leaf extract also from various suitable extractions and a linear anionic polysaccharide, which is useful at preventing opportunistic skin conditions including inflammation, acne-genesis, comedogenic environments and dryness, preferably in preadolescent and adolescent skin.

In certain embodiments, the invention encompasses the use of such combinations included in formulations intended as a skin care agents to help prevent poor skin conditions and help maintain clean, relatively bacteria free, non-irritated conditions of the dermis in pre-adult skin.

In certain embodiments, the N-acetylglucosamine is present in the composition in an amount of about 0.1% to about 5% (w/w) based on the total weight of the composition. In certain embodiments, the amount of N-acetylglucosamine is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% (w/w) based on the total weight of the composition.

In certain embodiments, the cholecalciferol is present in the composition in an amount of about 0.1% to about 5% (w/w) based on the total weight of the composition. In certain embodiments, the amount of cholecalciferol is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% (w/w) based on the total weight of the composition.

In certain embodiments, the tetrahexyldecyl ascorbate is present in the composition in an amount of about 0.1% to about 5% (w/w) based on the total weight of the composition. In certain embodiments, the amount of tetrahexyldecyl ascorbate is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% (w/w) based on the total weight of the composition.

In certain embodiments, the tetrahexyldecyl ascorbate is present in the composition in an amount of about 0.1% to about 5% (w/w) based on the total weight of the composition. In certain embodiments, the amount of tetrahexyldecyl ascorbate is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% (w/w) based on the total weight of the composition.

In certain embodiments, the resveratrol is present in the composition in an amount of about 0.1% to about 5% (w/w) based on the total weight of the composition. In certain embodiments, the amount of resveratrol is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10.0% (w/w) based on the total weight of the composition.

In certain embodiments, the white tea leaf extract is present in the composition in an amount of about 0.1% to about 5% (w/w) based on the total weight of the composition. In certain embodiments, the amount of white tea leaf extract is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10.0% (w/w) based on the total weight of the composition.

In certain embodiments, the linear anionic polysaccharide (e.g., Fucogel) is present in the composition in an amount of about 0.1% to about 5% (w/w) based on the total weight of the composition. In certain embodiments, the amount of linear anionic polysaccharide (e.g., Fucogel) is about 0.1, 0.2. 0.3, 0.4, 0.5, 0.6, 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0% (w/w) based on the total weight of the composition.

In certain embodiments, the first area of interest with untreated young skin is exfoliation and moisture balance. It is well known among the adult population that for years alpha and beta hydroxy acids have been used to treat the skin as an exfoliate and for a variety of dermatologic problems. Using these compounds, while effective, has been shown to have unwanted side effects of rendering the skin vulnerable to UV-B damage as well as innate irritability to the skin of the compounds themselves. This is unwanted in adult skin, much less on younger nascent skin, which is the application of interest in this disclosure. For these reason other compounds have been researched as viable substitutes. One particular molecule is N-acetyl glucosamine (NAG). NAG is an amino sugar of biological importance. It is a subunit in the building block of a bacterial cell wall and has been shown to have success treating certain immune disorders. Dermatology and cosmetology studies of NAG show the molecule is efficacious in moisturizing the skin, increasing cell turn over, and decreasing skin flaking. Studies show it is a viable and desirable replacement for stronger more irritating hydroxy-acids used for necessary exfoliation and moisture balance of the dermis. In certain embodiments, the formulations include a combination with other compounds of significance as a more appropriate treatment than any available means for pre-adolescent skin, as well as its use compared to its common counter parts.

In certain embodiments, the formulations include vitamin C in our formula, among other compounds of importance described herein. Ascorbic Acid is the most commonly known, water soluble, form of vitamin C and has long been used in dermatology and cosmetic skin care formulations. It has topical significance as a bactericidal agent, promotes collagen formation and is a potent antioxidant, protecting the skin from oxidation of many lipids, a process which leads to irritation and infection. In certain embodiments, the formulations include fat-soluble form of ascorbic acid, tetrahexyldecyl ascorbate, is the most stable, potent and efficaciously delivered unit of vitamin C into the dermis and most effective for young preadolescent skin in moderate dosing, as described in further embodiments in this disclosure. Tetrahexyldecyl ascorbate is an analogue of the commonly used L-Ascorbic Acid.

In certain embodiments, the formulations include Vitamin D. Vitamin D is essential to all human life and foremost health. The skin is the only organ capable of production of vitamin D. Vitamin D is present in very few food sources. It is available in two forms: D2 and D3. D2 is ergocalciferol and D3 is cholecalciferol. Cholecalciferol, D3, is the form of vitamin D synthesized in human skin for use. It is synthesized through direct exposure to UV light. Its presence in the skin is directly related to management of autoimmune disease, inflammatory disease and its absence is directly linked to psoriasis, acne, and rosacea among other common and potentially debilitating skin diseases that are seen at the onset of puberty and thereafter. In certain embodiments, the formulations include cholecalciferol, which is dose dependent in anti-proliferative action of skin cells, correlating its presence and absence with increased or reduced, respective, risks of skin cancer. Vitamin D in its most useful form, D3 greatly reduces the risk of skin infections as well. The abundant use of sunscreens, particular in those of younger ages has caused vitamin D deficiency increases in the population. Delivering vitamin D3 to the skin of preadolescents is of importance in preventing a variety of common popular health risks which may begin at or around the ages before the onset of puberty.

In certain embodiments, the formulations include antioxidants such as polyphenols are essential for control of skin health in the environment. A variety of substances including extracts like resveratrol and those from white tea leaf contain polyphenols which actively inhibit oxidation. Oxidation of lipids in various places in the dermis is a complex and long schematic which leads to acne and inherent inflammation in the skin⁴. Dietary nutrients such as vitamins C and E are important to mitigate such pathways leading to inflammation and skin infections as are polyphenols. Polyphenols are found in abundance in the extracts of leaves and skins of fruits and vegetable, such as those containing resveratrol and those stemming from white tea leaves. We propose herein that these two preferred sources of polyphenols are crucial in the preventative treatment of acne and have very important non-comedogenic effects in addition.

In certain embodiments, the formulations include a biosaccharide unit is implemented to give the skin a barrier protection to adhesion of bacteria and the departure of moisture from the skin. These are natural compounds of great biological activity and positive attributes for the skin, particular preadolescent skin which is focused on in this disclosure as the main type of application for prevention of acne and comedogens as well as providing for general optimal health and maintaining composition.

In certain embodiments, the compositions employed in the inventive methods and compositions are liquid, and thus the invention is effective even in the absence of the carrier. However, the compositions according to the invention comprise a cosmetically acceptable vehicle to act as a diluent, dispersant or carrier, so as to facilitate their distribution when the composition is applied to the skin.

The vehicle may be aqueous, anhydrous or an emulsion. Preferably, the compositions are aqueous or an emulsion, especially water-in-oil or oil-in-water emulsion. Water when present will be in amounts which may range from 5 to 99%, preferably from 40 to 90%, optimally between 60 and 90% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 and 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are: (1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate. (2) Ether-esters such as fatty acid esters of ethoxylated fatty alcohols. (3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. (4) Wax esters such as beeswax, spermaceti, myristyl myristate, stearyl stearate and arachidyl behenate. (5) Sterols esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

### Additional Skin Benefit Agents

Various types of additional active ingredients may be present in compositions of the invention. Actives are defined as skin benefit agents other than emollients and other than ingredients that merely improve the physical characteristics of the composition. Although not limited to this category, general examples include additional anti-sebum ingredients such as talcs and silicas, as well as alpha-hydroxy acids, beta-hydroxy acids, poly-hydroxy acids, benzoyl peroxide, zinc salts, and sunscreens.

Beta-hydroxy acids include salicylic acid, for example. Zinc pyrithione is an example of zinc salts useful in the compositions of the present invention.

Sunscreens include those materials commonly employed to block ultraviolet light. Illustrative compounds are the derivatives of PABA, cinnamate and salicylate. For example, avobenzophenone (Parsol 1789) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation.

Many cosmetic compositions, especially those containing water, must be protected against the growth of potentially harmful microorganisms. Anti-microbial compounds, such as triclosan, and preservatives are, therefore, necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

### Use of the Novel Compounds and Compositions

The compounds and compositions according to the invention are intended primarily as a product for topical application to human skin, especially as an agent for controlling or preventing excessive sebum secretion. Suppression of sebum provides multiple benefits, including improved skin condition; reduction of an unpleasant appearance and feel of greasy skin; reduction and/or prevention of acne, rosacea, seborrhea, oily scalp, oily/greasy hair, and dandruff.

In use, a quantity of the composition, for example from 1 to 100 ml, is applied to exposed areas of the skin, from a suitable container or applicator and, if necessary, it is then spread over and/or rubbed into the skin using the hand or fingers or a suitable device.

The present invention also includes a cosmetic method of controlling or preventing an oily skin condition, especially in the facial area, by applying to the skin the inventive composition. In another aspect, the present invention includes a cosmetic method of controlling, preventing, or treating oily or greasy hair.

The invention also includes a cosmetic method of reducing, preventing or controlling sebum secretion from sebocytes by applying the inventive composition.

The inventive methods and compositions provide control of sebum secretion from sebocytes, improved oil control and improved skin feel, and prevent shine and stickiness, while also providing anti-microbial activity against bacteria associated with acne and, generally, controlling microbial activity of bacteria on the skin surface, as well as providing anti-aging benefits which result in reduced appearance of wrinkles and aged skin, improved skin color, treatment of photoaged skin, improvement in skin's radiance and clarity and finish, and an overall healthy and youthful appearance of the skin. Additionally, the inventive methods are useful for reducing or preventing secretion from the apocrine glands.

In various embodiments, the invention includes compositions that are formulated with a pH that is acceptable to provide relief of disorders of the skin. In certain embodiments, the pH is about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.5, 7.6, 7.7, 7.8, or about 7.9.

In various embodiments, the cosmetic products are designed to integrate a variety of attributes, including essential functions (e.g. helping the skin appear vibrant, providing moisture retention), taste preferences (e.g. fragrance, color), usability, and so forth. In various embodiments, the compositions include viscosity measurements to control factors associated with achieving desired application comfort.

In certain embodiments, viscosity can impact a cosmetic product's chemical stability in its packaging over time. Therefore, given the many reasons why viscosity is important to the overall quality of cosmetic products, measuring viscosity has become key to successful cosmetic production companies.

In certain embodiments, the quality control of a product aims to verify if all of these defined features are in accordance with the standard definitions and if it will be maintained during the shelf life of the product. In certain embodiments, the quality control of cosmetics is important to ensure the efficacy and safety of products and its raw-materials. efficient, low cost and rapid methods to assay cosmetics' quality control are a priority owing to the explosive growth that cosmetic industries have witnessed worldwide. Rheology—which studies the flow and deformation of fluids—can be applied by the cosmetic industry to the evaluation of cosmetics' quality control in an efficient manner.

Rheology has been widely used because, by means of this tool, the researcher can determine physicochemical properties of a product. Specifically, in relation to the quality control of cosmetics, specifically, rheology can be applied to help in determining the stability of products by means of the apparent viscosity measured periodically in a determined period exposing the samples to stress conditions (high and low temperatures, solar irradiation), and to monitor the flow characteristics during the shelf life or in the stability assay of a product. The majority of cosmetic industries use viscometers to guarantee that the viscosity of different batches of a product is maintained. In addition, the rheology can be used to evaluate the stability over the time by dynamic and oscillatory rheological measurements and the release of active principles. Additionally, viscosity measurement of raw materials for cosmetic products are also vital for quality control.

In certain embodiments, the release profile, permeation, and transdermal absorption of topically administered compositions depends on the rate of release and the permeability of them into the skin and also of the viscosity of the formulation.

Viscosity and thickening agents are an important element of cosmetic formulation, offering the opportunity to change viscosity, improve rheological properties and modify the product aesthetics linked to consumer perception. Consistency is a textural property related to the amount of product felt between fingertip and skin. It relates to the ‘firmness’, ‘thickness’ or ‘viscosity’ of a liquid or fluid semi-solid. The higher the index of viscosity, the more resistant to withdrawal is the sample. This is an indicator of the cohesiveness and also the consistency or viscosity of the sample.

Viscosity control is especially effective in product control related to the application sensation of creams and emulsions. Emulsions are liquids which contain water droplets dispersed in oil or oil droplets dispersed in water and are a typical type of substance from the rheological point of view. Creams are a category of emulsions with special properties where the application of a certain amount of force results in a rapid fall in viscosity. What this means is that when the cream is applied to the skin, it becomes more spreadable. The normal high viscosity of the cream changes instantly to a lower viscosity when subjected to a force which allows it to be spread easily while providing a sensation of smoothness.

In certain embodiments, the invention encompasses compositions with a viscosity range of about 0.5 cP (centipoise) to about 50,000 cP. In certain embodiments, the viscosity is about 0.5, 50, 100, 500, 1000, 2500, 5000, 7500, 10,000, 12,500, 15,000, 17,500, 20,000, 22,500, 25,000, 27,500, 30,000, 32,500, 35,000, 40,000, 45,000, or about 50,000 cP.

### Product Form and Packaging

The cosmetic skin composition of the invention can be in any form, e.g. formulated as a toner, gel, lotion, a fluid cream, or a cream. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined to avoid contamination or degradation.

### EXEMPLARY FORMULATIONS OF THE INVENTION

General Formulation

| | | |
|---|---|---|
| | Int'l Nomenclature Cosmetic | |
| Ingredient | Ingredient | % (w/w) |

Phase A

| | | |
|---|---|---|
| Deionized Water | | 50-75% |
| Glycerin | Glycerin | 0.1-10% |
| Keltrol CG-T | Xanthan Gum | 0.1-1% |
| White Tea BT | Camellia Sinensis (White Tea) | 0.01-10% |
| | Extract | |
| Resveratrol BT | Resveratrol | 0.01-10% |
| Dermofeel PA-12 | Sodium Phytate | 0.01-0.5% |
| Carbopol 940 | Carbomer | 0.1-1% |
| Pemulen TR-2 | Acrylates/C10-30 Alkyl Acrylate | 0.1-1% |
| | Crosspolymer | |
| N-Acetyl Glucosamine | Acetyl Glucosamine | 1-5% |

Phase B

| | | |
|---|---|---|
| Fucogel 1.5P | Biosaccharide Gum-1 | 0.1-5% |

Phase C

| | | |
|---|---|---|
| Dermol DIA | Diisopropyl Adipate | 0.25-20% |
| DC 1403 | Dimethicone and Dimethicone | 0.25-20% |
| | Copolyol | |

Phase D

| | | |
|---|---|---|
| KOH Pellets | Potassium Hydroxide | 0.1-1% |
| Citric Acid | Citric Acid | 0.1-1% |

Phase E

| | | |
|---|---|---|
| Diocide | Phernoxyethanol and Caprylyl | 1-2% |
| | Glycol and Hexylene Glycol | |
| BVOSC | Tetrahexyldecyl Ascorbate | 0.1-5% |
| Vit D3 in Corn Oil | Cholecalciferol in Zea Mays | 0.01-0.5% |
| | (Corn) Oil | |
| SD 40B | Alcohol Denat. | 0-20% |

Phase F

| | | |
|---|---|---|
| Timiron Splendid Blue | Titanium Dioxide (and) Mica | 0.01-10% |
| | (and) Silica | |
| FD&C Red #33 (1% in | | 0.01-0.5% |
| Glycerin) | | |
| FD&C Blue #1 (1% in | | |
| Glycerin) | | |
| | Total | 100.00 |

Formulation No. 1

| | Int'l Nomenclature Cosmetic | % |
|---|---|---|
| Ingredient | Ingredient | (w/w) |
| Deionized Water | | 73.13 |
| Glycerin | Glycerin | 5.00 |
| Keltrol CG-T | Xanthan Gum | 0.20 |
| White Tea BT | Camellia Sinensis (White Tea) | 2.00 |
| | Extract | |
| Resveratrol BT | Resveratrol | 2.00 |
| Dermofeel PA-12 | Sodium Phytate | 0.10 |
| Carbopol 940 | Carbomer | 0.30 |
| Pemulen TR-2 | Acrylates/C10-30 Alkyl Acrylate | 0.20 |
| | Crosspolymer | |
| N-Acetyl Glucosamine | Acetyl Glucosamine | 1.00 |
| Fucogel 1.5P | Biosaccharide Gum-1 | 2.00 |
| Dermol DIA | Diisopropyl Adipate | 3.00 |
| DC 1403 | Dimethicone and Dimethicone | 2.00 |
| | Copolyol | |
| KOH Pellets | Potassium Hydroxide | 0.50 |
| Citric Acid | Citric Acid | 0.20 |
| Diocide | Phernoxyethanol and Caprylyl | 1.00 |
| | Glycol and Hexylene Glycol | |
| BVOSC | Tetrahexyldecyl Ascorbate | 0.10 |
| Vit D3 in Corn Oil | Cholecalciferol in Zea Mays (Corn) | 0.10 |
| | Oil | |
| SD 40B | Alcohol Denat. | 5.00 |
| Timiron Splendid Blue | Titanium Dioxide (and) Mica (and) | 2.00 |
| | Silica | |
| FD&C Red #33 (1% in | | 0.16 |
| Glycerin) | | |
| FD&C Blue #1 (1% in | | 0.01 |
| Glycerin) | | |
| | | 100.00 |

Formulation No. 2

| Ingredient | % (w/w) |
|---|---|
| Deionized Water | 54.00 |
| Glycerin | 5.00 |
| Keltrol CG-T | 0.20 |
| White Tea BT | 2.00 |
| Resveratrol BT | 2.00 |
| Algogen 2000 | 2.00 |
| Versene 220 | 0.10 |
| Dermofeel PA-12 | x |
| Carpopol 940 | 0.40 |
| Pemulen TR-2 | 0.30 |
| N-Acetyl Glucosamine | x |
| Fucogel 1.5P | x |
| Dermol DIA | 3.00 |
| DC 1403 | 5.00 |
| KOH Pellets | 0.50 |
| Diocide | 1.00 |
| BVOSC | |
| Vit D3 in Corn Oil | |
| Tagra Chameleon Brown | 5.00 |
| Deionized Water | 13.00 |
| SD 40B | 5.00 |
| Colorona Patagonian Purple | x |
| Colorona Mica Black | x |
| Colorona Blackstar Blue | 1.50 |
| Colorona Magenta | |
| | 100.00 |

Formulation No. 3

| Ingredient | % (w/w) |
|---|---|
| Deionized Water | 60.70 |
| Glycerin | 5.00 |
| Keltrol CG-T | 0.20 |
| White Tea BT | 2.00 |
| Resveratrol BT | 2.00 |
| Algogen 2000 | 2.00 |
| Versene 220 | 0.10 |
| Dermofeel PA-12 | x |
| Carpopol 940 | 0.30 |
| Pemulen TR-2 | 0.20 |
| N-Acetyl Glucosamine | x |
| Fucogel 1.5P | x |
| Dermol DIA | 3.00 |
| DC 1403 | 2.00 |
| KOH Pellets | 0.50 |
| Diocide | 1.00 |
| BVOSC | |
| Vit D3 in Corn Oil | |
| Tagra Chameleon Brown | 3.00 |
| Deionized Water | 13.00 |
| SD 40B | 5.00 |
| Colorona Patagonian Purple | x |
| Colorona Mica Black | x |
| Colorona Blackstar Blue | x |
| Colorona Magenta | 1.00 |
| | 100.00 |

Formulation No. 4

| Ingredient | % (w/w) |
|---|---|
| Deionized Water | 61.70 |
| Glycerin | 5.00 |
| Keltrol CG-T | 0.20 |
| White Tea BT | 2.00 |
| Resveratrol BT | 2.00 |
| Algogen 2000 | 2.00 |
| Versene 220 | 0.10 |
| Dermofeel PA-12 | x |
| Carpopol 940 | 0.30 |
| Pemulen TR-2 | 0.20 |
| N-Acetyl Glucosamine | x |
| Fucogel 1.5P | x |
| Dermol DIA | 3.00 |
| DC 1403 | 2.00 |
| KOH Pellets | 0.50 |
| Diocide | 1.00 |
| BVOSC | |
| Vit D3 in Corn Oil | |
| Tagra Chameleon Brown | 2.00 |
| Deionized Water | 13.00 |
| SD 40B | 5.00 |
| Colorona Patagonian Purple | x |
| Colorona Mica Black | x |
| Colorona Blackstar Blue | x |
| Colorona Magenta | 2.00 |
| | 100.00 |

Formulation No. 5

| Ingredient | % (w/w) |
|---|---|
| Deionized Water | 60.70 |
| Glycerin | 5.00 |
| Keltrol CG-T | 0.20 |
| White Tea BT | 2.00 |
| Resveratrol BT | 2.00 |
| Algogen 2000 | 2.00 |
| Versene 220 | 0.10 |
| Dermofeel PA-12 | x |
| Carpopol 940 | 0.30 |
| Pemulen TR-2 | 0.20 |
| N-Acetyl Glucosamine | 1.00 |
| Fucogel 1.5P | x |
| Dermol DIA | 3.00 |
| DC 1403 | 2.00 |
| KOH Pellets | 0.50 |
| Diocide | 1.00 |
| BVOSC | |
| Vit D3 in Corn Oil | |
| Tagra Chameleon Brown | 2.00 |
| Deionized Water | 13.00 |
| SD 40B | 5.00 |
| Colorona Patagonian Purple | x |
| Colorona Mica Black | x |
| Colorona Blackstar Blue | x |
| Colorona Magenta | 2.00 |
| | 100.00 |

Formulation No. 6

| Ingredient | % (w/w) |
|---|---|
| Deionized Water | 60.50 |
| Glycerin | 5.00 |
| Keltrol CG-T | 0.20 |
| White Tea BT | 2.00 |
| Resveratrol BT | 2.00 |
| Algogen 2000 | 2.00 |
| Versene 220 | 0.10 |
| Dermofeel PA-12 | x |
| Carpopol 940 | 0.30 |
| Pemulen TR-2 | 0.20 |
| N-Acetyl Glucosamine | 1.00 |
| Fucogel 1.5P | x |
| Dermol DIA | 3.00 |
| DC 1403 | 2.00 |
| KOH Pellets | 0.50 |
| Diocide | 1.00 |
| BVOSC | 0.10 |
| Vit D3 in Corn Oil | 0.10 |
| Tagra Chameleon Brown | 2.00 |
| Deionized Water | 13.00 |
| SD 40B | 5.00 |
| Colorona Patagonian Purple | x |
| Colorona Mica Black | x |
| Colorona Blackstar Blue | x |
| Colorona Magenta | 2.00 |
| | 100.00 |

Formulation No. 7

| Ingredient | % (w/w) |
|---|---|
| Deionized Water | 62.50 |
| Glycerin | 5.00 |
| Keltrol CG-T | 0.20 |
| White Tea BT | 2.00 |
| Resveratrol BT | 2.00 |
| Algogen 2000 | x |
| Versene 220 | x |
| Dermofeel PA-12 | 0.10 |
| Carpopol 940 | 0.30 |
| Pemulen TR-2 | 0.20 |
| N-Acetyl Glucosamine | 1.00 |
| Fucogel 1.5P | 2.00 |
| Dermol DIA | 3.00 |
| DC 1403 | 2.00 |
| KOH Pellets | 0.50 |
| Diocide | 1.00 |
| BVOSC | 0.10 |
| Vit D3 in Corn Oil | 0.10 |
| Tagra Chameleon Brown | x |
| Deionized Water | 13.00 |
| SD 40B | 5.00 |
| Colorona Patagonian Purple | x |
| Colorona Mica Black | x |
| Colorona Blackstar Blue | x |
| Colorona Magenta | x |
| | 100.00 |

The following specific examples further illustrate the invention, but the invention is not limited thereto.

### Example 1

### Procedure:

In main vessel prepare Phase A by adding water.

Disperse Carbopol 940 and Pemulen TR-2. Mix until uniform.

Dissolving N-Aectyl Glucosamine, Algogen 2000 then remaining Phase A ingredients.

In separate vessel combine Phase B ingredients—Fucogel. Mix until uniform and add to batch.

Add Phase C—DC 1403

Add Phase D—KOH. Mix until uniform and free from lumps. Homogenize.

Stop homogenizing and add Phase E.

In separate vessel combine Phase F ingredients in order listed.

Mix with low shear until uniform and powders are wet out.

Add to batch with planetary mixing in main vessel.

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

1. A skin care composition comprising:
N-acetylglucosamine (NAG), cholecalciferol, tetrahexyldecyl ascorbate, resveratrol, white tea leaf extract, and an anionic polysaccharide.

2. The skin care composition of claim 1, wherein the N-acetyl-glucosamine is present in an amount of about 1% (w/w) to about 5% (w/w) based on the total weight of the composition.

3. The skin care composition of claim 1, wherein the cholecalciferol is present in an amount of about 0.1% (w/w) to about 5% (w/w) based on the total weight of the composition.

4. The skin care composition of claim 1, wherein the tetrahexyldecyl ascorbate is present in an amount of about 0.1% (w/w) to about 5% (w/w) based on the total weight of the composition.

5. The skin care composition of claim 1, wherein the resveratrol is present in an amount of about 0.1% (w/w) to about 10% (w/w) based on the total weight of the composition.

6. The skin care composition of claim 1, wherein the white tea leaf extract is present in an amount of about 0.1% (w/w) to about 10% (w/w) based on the total weight of the composition.

7. The skin care composition of claim 1, wherein the linear anionic polysaccharide is present in an amount of about 0.1% (w/w) to about 5% (w/w) based on the total weight of the composition.

8. The skin care composition of claim 1, wherein the amount of NAG is about 1.0% (w/w) based on the total weight of the composition.

9. The skin care composition of claim 1, wherein the amount of cholecalciferol is about 0.1% (w/w) based on the total weight of the composition.

10. The skin care composition of claim 1, wherein the amount of tetrahexyldecyl ascorbate is about 0.1% (w/w) based on the total weight of the composition.

11. The skin care composition of claim 1, wherein the amount of resveratrol is about 2.0% (w/w) based on the total weight of the composition.

12. The skin care composition of claim 1, wherein the amount of white tea leaf extract is about 2.0% (w/w) based on the total weight of the composition.

13. The skin care composition of claim 1, wherein the amount of linear anionic polysaccharide is about 2.0% (w/w) based on the total weight of the composition.

14. The skin care composition of claim 1, wherein the linear anionic polysaccharide is biosaccharide gum-1.

15. The skin care composition of claim 1, comprising the following components:
(i) about 1.0% (w/w) NAG;
(ii) about 0.1% (w/w) cholecalciferol;
(iii) about 0.1% (w/w) tetrahexyldecyl ascorbate;
(iv) about 2.0% (w/w) resveratrol;
(v) about 2.0% (w/w) tea leaf extract; and
(vi) about 2.0% (w/w) anionic polysaccharide.

16. A method of treating or preventing a skin condition comprising administering a skin care composition of claim 1.
